(19) United States
(12) Patent Application Publication
(10) Pub. No.: US 2021/0228266 A1
(43) Pub. Date: Jul. 29, 2021

(54) MINIMALLY INVASIVE DEVICE AND METHOD FOR TIGHTENING SAGGING SKIN BY LINEAR TENSING AND STIMULATION OF COLLAGEN PRODUCTION, WHEREIN THE ANESTHESIA, HEAT, AND ADDITIONAL COLLAGEN INDUCTION OR ANTI-INFLAMMATORY FLUIDS CAN BE APPLIED WITH THE SAME APPARATUS AND IN THE SAME AREA

(71) Applicant: YAE, LLC, Coral Gables, FL (US)

(72) Inventor: Fernando Benjamin FISCHMANN, Vitacura Santiago (CL)

(21) Appl. No.: 17/154,686

(22) Filed: Jan. 21, 2021

Related U.S. Application Data
(60) Provisional application No. 62/964,961, filed on Jan. 23, 2020.

(51) Int. Cl. A61B 18/14; A61B 18/12

(52) U.S. Cl. CPC A61B 18/1477; A61B 2018/00017; A61B 18/1206

(57) ABSTRACT

The invention provides an innovative low cost, efficient, short duration, and painless minimally invasive device and method for tightening sagging skin through linear tensing and stimulation of collagen production. More particularly still, the device and method not only allow applying heat directly in the dermis layer of the skin, but also allows for administering an anesthetic and optionally additional ant-inflammatory or collagen induction fluids through the same apparatus.

### FIELD OF THE INVENTION

The present invention relates generally to the field of minimally invasive cosmetic procedures for improving the laxity of the skin and to delay the signs of aging in humans. More particularly, the invention provides an innovative low cost, efficient, short duration, and painless minimally invasive device and method for tightening sagging skin through linear tensing and stimulation of collagen production. More particularly still, the device and method not only allow applying heat directly in the dermis layer of the skin, but also allows for administering an anesthetic and optionally additional anti-inflammatory or collagen induction fluids through the same apparatus.

### BACKGROUND

A desire of a youthful appearance has become common in our society and has resulted in an improvement in cosmetic procedures aiming to reverse the aging process. Throughout the latter part of the 20th Century, advances in medicine and nutrition, combined with an increasing awareness of individual healthcare, have enabled people to live longer, healthier, and in more productive way. As the population ages, more people are seeking ways to enhance their appearance for personal and professional reasons. Beauty and youth became significant determinants of economic security, and in order to appear well groomed and confident as well as to remain viable in the workplace, middle aged men and women, began seeing the growing specialty of cosmetic surgery as a solution to the natural signs of aging.

As people age, their concerns about their appearance mainly focuses on the face. Studies have indicated that while younger men and women were mostly concerned about the shape and appearance of their bodies, older individuals were worried about their faces. In particular, the older groups disliked wrinkles and drooping skin. Research also shows that 4 out of 5 American women follow beauty industry trends and regularly use fragrance products, and according to the American Society of Plastic Surgeons (ASPS), almost 18 million people underwent surgical and minimally invasive cosmetic procedures in the United States in 2018.

The foregoing explains why the beauty industry has grown exponentially in the last decades to become today a global business that is worth more than USD 500 billion, and while projections for growth vary, most agree it will continue to advance at a 5%-to-7% compound-annual-growth-rate to reach or exceed $800 billion by 2025 in the world.

Therefore, there is a need in the art for a device and method that provides a minimally invasive cosmetic procedure for improving laxity of the skin and to delay the signs of aging in humans that addresses limitations in available devices and procedures.

### SUMMARY

A preferred embodiment in accordance with the principles of the present invention provides for a device and method that is minimally invasive directed to cosmetic procedures for improving the laxity of the skin and delaying the signs of aging in humans. In particular, the invention provides an innovative low cost, efficient, short duration, and painless minimally invasive device and method for tightening sagging skin through linear tensing and stimulation of collagen production, wherein the apparatus not only allows applying heat directly in the dermis layer of the skin, but also allows administering an anesthetic and optionally additional treatment fluids through the same apparatus and in the same area. In this manner many of the limitations of the existing devices and procedures are overcome.

While those of skill in the art may have an understanding of causes and existing treatments for improving laxity of the skin and delaying signs of aging, a brief description is next provided.

Intrinsic aging processes include loss of skin elasticity and collagen, along with fat atrophy. Extrinsic factors, such as solar radiation, damage the skin, with impacts on collagen and elastic fibers. Other factors that can contribute to an aged appearance to the face include general poor health, an unhealthy diet, cigarette smoking, and alcohol. The main and often most perceived signs of aging include a lined forehead, drooping brows, loss of cheek roundedness, sagging neck lines, loss of chin definition, drooping of the nasal tissues, and wrinkling of the skin around the mouth.

In order to understand the aging process, it is important to bear in mind that skin is made of two main layers: the outer one is the epidermis, made mainly by keratinocytes, responsible for the formation of a barrier against environmental damages (e.g., pathogens, heat, UV radiation and water loss). The inner layer, the dermis, comprises connective tissue, made by structural components such as collagen (responsible for the skin firmness), elastic fibers (responsible of the skin elasticity) and extracellular matrix (structural component). A third layer of subcutaneous tissues contains fat cells that provide insulation to the body.

The formation of skin wrinkles and subsequent skin sagging is triggered by a failure of the skin structures from a lack of collagen or to its changes, like thinning and/or fractioning, caused by the stretching and repeated extension of some areas of the skin, especially the face, on which the lack of elastin has an important role. Its decrease and subsequent loss of elasticity, causes an increase of volume of the skin and issues such as the double chin. All these combined changes in the scaffolding of the skin cause the appearance of wrinkles, the shape and form of which depends on the nature of associated skin and muscle contraction. The consequences of this degenerative process lead to enhanced skin fragility and a decrease of the amount of nutrients available to the epidermis. These consequences interfere with the normal skin repair process, therefore causing more noticeable wrinkling and sagging. In addition, by making facial expressions regularly, muscles permanently contract causing the skin to wrinkle due to loss of tissues as described above. Moreover, the effects of gravity are also responsible for the appearance of wrinkles and skin sagging. This promotes jowls and drooping eyelids.

Aging as such, is an irreversible occurrence that affects the skin due to a decrease of structural substances included in the layers or in imperfect remodeling of the fibers (mainly collagen) and other multifactorial issues that cause the formation of wrinkles. Regeneration of the lost tissues, including mainly collagen fibers and elastin, is therefore a critical target for wrinkle treatment and prevention.

In order to reduce sagging of skin and wrinkles, there are two main categories for different types of lifting techniques: (A) Surgical lifts, and (B) non-surgical lifts.

Surgical lifts, such as eyelid surgery, face lifts, and forehead lifts have seen a more than 50% decrease over the last 18 years, while non-surgical facial rejuvenation procedures continue to experience growth, with more than USD 6.8 billon spent on non-surgical skin rejuvenation in 2016 in the U.S. alone.

Non-surgical facial rejuvenation, although it has less dramatic results than surgical options, has grown due to its advantages related to less downtime, lower costs, less risky, and shorter duration than surgical procedures.

Today, there are three main types of non-surgical treatments that can be applied in order to reduce visible wrinkles and sagging skin: (i) subtle tightening, (ii) non-invasive (without skin penetration) and (iii) minimally invasive treatments (with skin penetration).
i. Subtle tightening
   A subtle superficial tightening can be achieved by means of skin-firming creams and lotions that provide mainly a moisturizing effect into the skin that can plump up the epidermis, making fine lines and wrinkles less noticeable. Some cosmetic products incorporate substances such as retinol that can help in the production of collagen. However, the results of skin-firming creams and lotions are very subtle and temporary.
   Other cosmetic non-surgical treatments such as the use of steam, exfoliation, extraction, facial masks, peels, and massage also exert an influence on the epidermis (the outer layer of the skin) and therefore their effect might not have a substantial impact on the appearance of skin or in the reduction of visible wrinkles, having only a subtle result.
ii. Non-invasive skin tightening procedures with production of heat from the outside that passes through the epidermis in order to reach the inner layer of the skin (dermis)
   These procedures are called non-invasive because they do not require making a puncture wound or incision. However, these procedures might create redness and swelling on the outer layer of the skin, which can last several days.
   Non-invasive skin-tightening procedures allow for results that tend to appear gradually, so they seem natural and usually they take about one hour or less to be completed. These treatments are focused on generating a “controlled damage” of the skin, to boost skin regeneration for example.
   The most common non-invasive skin tightening procedures are ultrasound, radiofrequency and laser procedures, where each of these types of procedures consists of applying heat or irradiating heat to the skin from the outside layer (epidermis) to boost the production of collagen. Therefore, while these procedures aim at directing the heat to the dermis, the treatments are performed from the outside (from the epidermis), generally by means of a device that is placed against the skin. Accordingly, the heat passes through the epidermis in order to reach the inner layer of the skin (dermis). With one treatment, most people see modest lifting with limited results within 2 to 6 months.
iii. Minimally invasive skin tightening procedures through heat irradiation or direct heat application in the dermis
   There are two types of minimally invasive skin tightening procedures that rely on heat applied or irradiated directly to the dermis, specifically:
   i) Laser treatments that aim at producing laser beams that penetrate the skin and can reach the inner layer of the skin (dermis) to heat it directly, such as Fraxel® and Co2re® where laser beams are produced such that they penetrate the skin and reach the dermis layer. This type of treatment does not entail a physical penetration of the skin, but the laser beam penetrates without needing a puncture or incision.
   ii) Treatments where a device or apparatus is physically introduced through the epidermis in order to reach the dermis. For example, needles or probes are introduced in the skin in order to reach the dermis, and then the heat is irradiated or applied directly at the dermis layer.
   From these types of procedures that aim at heating the dermis layer directly, the ones that are more effective are the treatments where probes or needles are introduced through the epidermis to reach the dermis layer, which are able to provide a direct physical contact between the probe or needle with the dermis to provide heat irradiation or direct heat application. These procedures are believed to represent the state of the art in terms of this field.
   For example, when a probe is used, the probe itself can irradiate heat or apply heat directly to the dermis, as its material or configuration allows for it. For example, the material of a probe can be an electrode that produces a radiofrequency at the extreme or distal end of the probe through a direct cable/conductor connected to the probe.
   In comparison to the non-invasive skin tightening procedures (e.g., without skin penetration) that rely on applying heat to the outer layer of the skin to hopefully reach the inner layer to promote collagen denaturation (tightening), minimally invasive skin tightening procedures that can heat the dermis directly through insertion of probes or needles are able to provide better and more noticeable results since they produce a direct effect in the inner layers of the skin, while boosting localized collagen and elastin production.

It is important to highlight that most of the aforementioned procedures aim at irradiating the dermis through radiofrequency or applying heat directly to the dermis, which can boost the production of collagen and elastin in order to reduce wrinkling and sagging of the skin more efficiently. By applying heat directly in the dermis, collagen denaturation occurs. Denaturation of collagen is the phenomenon of thermal shrinkage of collagen, which begins with a denaturation of the triple helix of the collagen molecule. When collagen is heated, the heat-labile intramolecular cross-links are broken, and the protein undergoes a transition to a random, gel-like state (denaturation). Collagen shrinkage occurs through the cumulative effect of the “unwinding” of the triple helix, due to the destruction of the heat-labile intramolecular crosslinks, and the residual tension of the heat-stable intermolecular cross-links. Heated fibroblasts are also implicated in new collagen formation and subsequent tissue remodeling which can also contribute to the final cosmetic result. The precise heat-induced behavior of connective tissues and the extent of tissue shrinkage are dependent on several factors which include the maximum temperature reached, exposure time, tissue hydration and tissue age.

The big difference between treatments applied in the outer layer of the skin (epidermis) or inner layer of the skin (dermis) is that the ones that are able to heat the inner layer of the skin directly are more efficient, as they heat the zone where collagen is produced, instead of having to heat the outer layer (epidermis) in order to reach the inner layers of the skin, which causes redness and sometimes mild burning of the epidermis.

Therefore, treatments that aim at heating the dermis directly by using needles and probes that are in direct physical contact with the dermis are more efficient, and there are two main types in the market today that are based on the use of radiofrequency:

### A. Microneedling:

Microneedling refers to a treatment where a series of insulated small sized needles are used to penetrate the skin and release radiofrequency currents from the needle tips producing thermal zones in the dermal structural components. This process is known to trigger long-term dermal remodeling, neoelastogenesis, and neocollagenesis. The depth of the needles varies from 0.5 mm to 3.5 mm which allows targeting different layers of the dermis. In one example, the main radiofrequency delivery system has a disposable tip with 49 gold plated needles. This microneedling radiofrequency (MNRF) technology does not produce heat directly on the epidermis but on the dermis.

The microneedling treatment is a long treatment that generally commences with a local topic anesthetic being applied to the epidermis, such as an anesthetic cream that is applied and then produces a mild numbing effect generally within 30 to 45 minutes. Afterwards, anesthetic shots are administered to the area being treated, where the anesthetic (generally lidocaine) is applied by a simultaneous 3-point application syringe that administers anesthetic to three points at the same time. Such application is usually replicated throughout the entire skin surface being treated (such as the neck and face), at least 10 times. This means that, for example, 3 shots are administered at least 10 times, resulting in at least 30 painful anesthetic shots being applied to a patient. In other cases, simultaneous 5-point application syringes are used, increasing the amount of shots for the anesthesia application.

Therefore, this treatment takes a long time due to the application of the first topic anesthetic, and then for the additional simultaneous 3-point (or 5-point) anesthetic application that is applied to the skin surface.

In addition, since the microneedling device has 49 independent needles, each application of the device to the skin creates an additional 49 punctures that may produce pain. As an example, a typical cheek treatment requires more than 5 device applications, resulting in more than 245 punctures in the skin from the microneedles (not counting the previously applied anesthetic punctures on the skin).

Further, the anesthetic that is applied through the 3-point (or 5-point) simultaneous applicator syringe is not homogeneous. This in turn results in the numbness of the skin being non-homogeneous and can result in some applications of the microneedling device being painless and some being very painful. The non-homogeneity causes discomfort and tension in the patient vis-à-vis being worried if the next application of the microneedling device will be painful or not.

Additionally, the microneedling device works in a relatively random operation and does not produce lineal contractions of the skin, but only works on very specific application points where it is used and where the microneedle is located.

Finally, the device and equipment used to apply these microneedling techniques are sophisticated and expensive, resulting in expensive treatments that including the application of anesthetics can last generally of up to 2 hours and generally has a cost of between USD $3,000 to USD $5,000 depending on the area to be treated.

### B. Percutaneous Heating Probe:

This technique for face skin rejuvenation comprises the use of a probe that is introduced underneath the skin into the dermis layer in order to administer radiofrequency directly to the dermis and subdermal tissue through manual displacement of the probe around the face and neck areas (among other areas of the body).

This treatment requires introducing a probe that has a tip that irradiates heat through radiofrequency and therefore by moving the probe through the dermis, the probe's tip irradiates the dermis layer through radiofrequency and heats that local area. Subdermal temperatures are monitored and controlled by a thermistor integrated within the thermocoupled handpiece. Concurrently, epidermal temperatures are monitored using an infrared camera system. According to this treatment, both subdermal and dermal collagenous tissues reach therapeutic temperature thresholds necessary for collagen remodeling.

This treatment usually requires a first step of topic anesthetic application (similarly than in microneedling techniques), where a local topic anesthetic is applied to the epidermis, such as an anesthetic cream that is applied and then produces a mild numbing effect within 30 to 45 minutes.

Afterwards, anesthetic shots are administered to the area being treated, where the anesthetic (generally lidocaine) is applied by a simultaneous 3-point application syringe that allows to administer the anesthetic to three points at the same time, and such application is replicated throughout the entire skin surface being treated, at least 10 or even 20 to 30 times in the case of face and neck applications. In other cases, simultaneous 5-point application syringes are used, increasing the amount of shots for the anesthesia application.

Since the anesthetic that is applied through the 3-point (or 5-point) simultaneous applicator syringe is not homogeneous, which causes that the numbness of the skin is non homogeneous, this can result in some perforations and heat application from the probe to be very painful, which also generates discomfort and tension in the patient that is worried if the next application of the heating probe will be painful or not. This treatment also may take up to 2 hours including the time needed for the anesthetic to be applied.

Particularly for this type of treatment, since the probe is inserted and then is displaced and moved manually through the dermis (under the skin), the treatment may damage blood vessels or nerves that are located under the skin and generates an additional and important risk to the patient. Additionally, improper manipulation may also create severe burns.

The device and moving probe are also sophisticated and expensive systems, resulting in expensive treatments with a cost generally between USD $3,500 to USD $5,000 depending on the area to be treated.

Therefore, the two aforementioned techniques designed to deliver RF energy to the dermis layer of the skin apart from being expensive, are very painful and generally require lengthy treatment periods in order to provide a first anesthetic application that can be painful.

Other types of treatments that have not reached successful commercial application due to their complexity and high costs include the use of multiple implants that carry heater segments that are heated mainly through electromagnetism with very complex equipment, and that are inserted in the skin so that they are located in the interface between the dermis and the fatty layer of the skin, and aim at providing direct heating of the dermis layer of the skin to achieve a specific percentage of linear collagen shrinkage.

Therefore, even though commercial techniques to rejuvenate skin that utilize irradiated radiofrequency to generate heat in the dermis layer of the skin via micro needling or moving probes have proved to be more efficient than non-invasive treatments, they still show a number of drawbacks associated to such techniques.

Accordingly, there is a demand for cosmetic procedures to reduce the visible effects of such skin distortions including a large demand for “tightening” skin to remove sags and wrinkles especially in the regions of the face and neck. Furthermore, new systems are sought that can stimulate collagen production and subsequent tightening without causing visible damage to the epidermis of the skin, without requiring large application of topical and subcutaneous anesthetics, that are painless, have simple configurations, and that are inexpensive procedures that allow a controlled and linear heating of the dermis layer of the skin to achieve said collagen stimulation with improved and longer lasting results.

Therefore, according to one aspect of the invention, there is provided a low cost minimally invasive apparatus for tightening sagging skin by linear tensing and stimulation of collagen production, which allows to administer fluids such as anesthetics and optionally other treatment fluids through the same apparatus. The apparatus comprises an insertable energy transfer device having an elongated shape, wherein the device has a hollow body, a tip, and a base and includes at least one orifice located in the body or tip of the energy transfer device, through which an effective amount of an anesthetic and optionally other treatment fluids can be administered into a dermal layer of a patient and a temperature sensor located in the energy transfer device that measures the temperature of the dermis in immediate contact with the energy transfer device. The apparatus also includes an external generation device configured to generate energy that is transferred to the energy transfer device.

According to another aspect of the invention, there is provided a low cost minimally invasive linear tensing cosmetic method for tightening sagging skin by linear tensing and stimulation of collagen production by applying heat to the dermis layer of the skin directly, which comprises the following steps:
a) providing an energy transfer device with an elongated shape and a hollow body, comprising at least one orifice located within the body or tip of the energy transfer device;
b) inserting the energy transfer device into a dermal layer of a patient's skin;
c) administering an effective amount of anesthetic into a dermal layer of a patient through the at least one orifice located in the body or tip of the energy transfer device;
d) generating energy through an external generation device, wherein such energy is transferred into the energy transfer device in order to apply heat to the dermis layer of the skin;
e) measuring the temperature of the dermis that is in immediate contact with the energy transfer device through a temperature sensor located within the energy transfer device;
f) controlling the external generation device so that the energy transfer device allows to heat the dermis layer up to a temperature of between 45° to 75° C.;
g) maintaining the minimum temperature generated in the dermis in immediate contact with the energy transfer device for a time of up to 10 minutes;
h) cooling the patient's skin in the vicinity of the treatment area;
i) repeating steps c. through h. as necessary, taking advantage of the already introduced energy transfer device in contact with the dermis layer of the skin; and
j) removing the energy transfer device from the skin.

While the invention will be described with respect to preferred embodiment configurations and with respect to particular components and structures, it will be understood that the invention is not to be construed as limited in any manner by either such components or structures described herein. Instead, the principles of this invention extend to any method and apparatus in accordance with the claims.

These and other variations of the invention will become apparent to those skilled in the art upon a more detailed description of the invention. The advantages and features which characterize the invention are pointed out with particularity in the claims annexed hereto and forming a part hereof. For a better understanding of the invention, however, reference should be had to the drawings which form a part hereof and to the accompanying descriptive matter, in which there is illustrated and described a preferred embodiment of the invention.

### BRIEF DESCRIPTION OF THE DRAWINGS

Referring to the drawings, wherein like numerals represent like parts throughout the several views:

FIG. 1 shows a previous art plot of linear shrinkage versus time for various temperatures.

FIG. 2 shows a representative distribution of the main layers of the human skin (3) including the epidermis (4), the dermis (5) and the fat tissue (6).

FIG. 3 shows a first representative method (1) according to the principles of the present invention, wherein it is appreciated that steps 1c through 1h can be performed in a different order.

FIG. 4 shows a representative embodiment of the energy transfer device (7) wherein the device is hollow and includes a base (10), a tip (9), a heating element (8), and at least one orifice (12).

FIG. 5 shows another representative embodiment of the energy transfer device (7) wherein the device is hollow and includes a base (10), a tip (9), a heating element (8), and a plurality of orifices (12).

FIG. 6 shows a representative embodiment of a handheld device (2) comprising an energy transfer device (7) with a plurality of orifices (12). The external generation device (13) is also shown, as well as fluid storage elements (14) that are connected to the energy transfer device in order to allow administering treatment fluids contained within the fluid storage elements (14) to the dermis layer of the skin.

### DESCRIPTION OF THE INVENTION

The present invention relates to the field of minimally invasive cosmetic procedures for improving the laxity of the skin and to delay the signs of aging in humans. In particular, the invention provides an innovative low cost, relatively short procedure time, efficient, and painless minimally invasive device and method for tightening sagging skin using linear tensing and stimulation of collagen production.

The effects of dermal heating are well-recognized and include immediate effects on collagen structure with stimulation of dermal fibroblasts inducing a synthesis of new collagen fibers (known as neocollagenesis) and elastic fibers (known as neoelastogenesis).

The present invention allows heating the dermis layer of the skin in a controlled and efficient manner, thereby avoiding the limitations, problems and risks associated with current commercially applied solutions. The current solutions, as previously mentioned, usually require the use of a topical anesthetic that takes a long time to take effect. Then, a subcutaneous application of anesthetic step is performed, which requires many painful punctures in the treatment area and does not create a homogeneous effect. These drawbacks result in a long total treatment time, difficulty in repeating the treatment in the same location, requiring different devices and applications for the anesthesia (e.g., the anesthetic is not applied through the same treatment apparatus), and do not allow administering anti-inflammatory, anti-bruising, or other collagen induction fluids in the same point where the treatment is performed. Finally, the equipment and devices used for current treatments are very expensive and complex.

For example, in the case of current techniques such as microneedling and moving probe techniques, such techniques: have long anesthetic application and procedure duration; require a large number of punctures for anesthetic application; are prone to risks of moving a probe under the skin that can damage blood vessels or nerves or even generate serious burns (for moving probe techniques); may cause unexpected painful treatment due to non-homogeneous anesthetic application; do not have a lineal tensing effect; and have very high equipment costs with complex configurations.

Embodiments constructed in accordance with the present invention overcome the previous problems and limitations, and provide an innovative apparatus and method that improves the results of skin tightening by using heat that is directly applied to the dermis layer of the skin by having an energy transfer device that is inserted through the skin in order to reach the dermis layer and applies heat into such layer directly, allowing a precise temperature and application time control, as well as providing linear tensing effects, wherein in a very innovative manner compared to current treatments, the anesthetic is also applied directly through the apparatus in the same location that the treatment is being performed, representing a revolutionary and important change in this field that may generate a very big impact in the market. As an additional innovative and surprising effect, the treatment of the present invention can be repeated in the same location with different combinations of temperature and time, and can also include the administration of other treatment fluids such as anti-inflammatory, anti-bruising, or skin tightening treatment substances, which also contrasts with current techniques that do not encompass these steps.

It is important to mention that none of the abovementioned techniques, such as microneedling or moving probes, allow repeating the treatment in the same location, or applying anesthetics through the same apparatus, or administering other fluids such as anti-inflammatory, anti-bruising, or collagen induction fluids through the same apparatus and in the same location where the treatment was performed.

In particular, the apparatus and method of the invention aim to stimulate collagen and elastin to generate subsequent tightening of the skin without causing visible damage to the epidermis of the skin and without requiring a long and painful application of anesthetics, in a short, painless and inexpensive procedure that allows a controlled, linear tensing through heating of the dermis layer of the skin directly in order to achieve said collagen stimulation with improved and longer lasting results, overcoming the drawbacks from current technologies, as well as providing a new and innovative methods and apparatus that allow to administer not only anesthetics through the same device, but also allows to administer additional treatment fluids to the skin area that is being treated during the treatment process. Further, the method and apparatus of the present invention allow repeating the treatment in the same location as necessary, having many advantages over current treatment procedures.

Additionally, the method of the present invention allows evaluating the shrinkage rate and aesthetic view of the lineal tensing of the skin in real time, allowing the technician to determine if the treatment needs to be repeated in the same location and to determine the temperature and treatment time to be applied on a specific patient. This is an important feature as current techniques such as microneedling do not allow visually evaluating the shrinkage rate but those treatments have to wait several weeks to provide the full effect. None of the current commercially applied methods allow to visualize and evaluate the lineal shrinkage of the skin in real time in order to determine next steps of the treatment. It's important to note that this shrinkage rate and visualized shrinkage will be part of the total shrinkage rate achieved in the skin.

### Description of the Apparatus

The unique apparatus of the present invention allows heating the dermis layer of the skin directly, in order to tighten sagging skin by linear tensing and stimulation of collagen production. The apparatus according to the principles of the present invention not only addresses the drawbacks of minimally invasive procedures that are commercially used, such as microneedling or moving probes, but also in an innovative manner allows the technician to apply the anesthetic in a localized manner and in the precise spot where the heat is being applied. In addition, the apparatus according to the present invention can allow the administration not only of anesthetics previous or during the procedure, but also of treatment fluids such as anti-inflammatory, anti-bruising, or other collagen promotion fluids at any time during the treatment process.

An apparatus in accordance with the principles of the present invention comprises an energy transfer device having an elongated shape and that has a base and a body, wherein the length of its body is at least 1 cm, which allows the technician to insert the device in the patient's skin and to reach at least the dermal layer of the same. The energy transfer device has an elongated shape like a hollow cannula or needle that allows introducing a fluid substance through the same. The energy transfer device comprise anti-allergen materials to minimize risk of generating an allergic reaction when introduced to the patient's skin.

In an embodiment of the invention, the energy transfer device is a hollow cannula that is introduced through the skin by means of an introducing device or needle, typically used in cannulas. In another embodiment of the invention, the energy transfer device has a pointed tip for allowing its introduction to the skin, such as a needle or syringe. The needle or syringe may be constructed so that it can be sterilized by conventional sterilization techniques or can be disposable elements.

The energy transfer device may comprise a textured surface to achieve focalized energy transfer into the dermis layer of the skin. For example, the energy transfer device may comprise spiral or helicoidally configured protrusions that generate a spring-like effect on the skin when such spirals transfer energy into the dermis layer of the skin.

In another embodiment of the invention, the energy transfer device comprises elements or materials located in the inside or embedded into the energy transfer device, and that have an helicoidally configured pattern that cause heating through such elements or materials and therefore generates a spring-like effect on the skin when such materials or elements transfer energy into the dermis layer of the skin. In this embodiment, the materials or elements within the energy transfer device do not generate any protrusions or texture of the device.

In an embodiment of the invention, the energy transfer device comprise flexible materials so as to adapt to contours of the skin and/or to achieve easier introduction into the dermis layer of the skin, In another embodiment of the invention, the energy transfer device comprises rigid materials to maintain its shape and design while introduced to the skin.

Also, in an embodiment of the invention, double-walled cannulas or needles may be used.

In a preferred embodiment of the invention, at least a portion of the device comprises a heating element that is able to increase its temperature.

In a more preferred embodiment of the invention, the heating element can be an electrically conductive material that increases its temperature in response to an electrical current passing through a resistive element, coil, or by other similar means exhibiting electrical resistance. Such electrically conductive material can be used for more intense or less intense treatments, depending on its configuration. This embodiment requires a physical connection between the energy transfer device and external generation equipment capable of generating the current needed for increasing the temperature of the heating element in a controlled manner. It is important to note than when conductive materials are used, the energy transfer device is configured so that a portion of it acts as a thermal insulator in order to avoid the heat to be transmitted and conducted to other portions of the skin. In an embodiment of the invention, when using a cannula or similar device, such device is constructed so it has an insulated section that allows the circulation of electricity through the heating element. The energy transfer device and its heating element are configured to achieve a substantially homogeneous temperature across the introduced portion of the device.

Alternatively, the heating element can be an electromagnetically inducible material, which means that is able to increase its temperature in response to an electromagnetic field in its vicinity, also known as induction heating. For the purposes of the present embodiment of the invention, the electromagnetically inducible material that covers at least a portion of the energy transfer device is a ferromagnetic material with a Curie temperature of at least 300 K.

When electromagnetically inducible material is used, the inducer comprises equipment and systems that are placed over the skin or surrounding the skin where the energy transfer device is introduced in order to achieve a substantially homogeneous temperature of the energy transfer device in such zone.

The heating element is configured so that it allows applying a temperature of between 45 and 75 degrees Celsius for a time range depending on the treatment intensity, with a maximum application time of 10 minutes, which can be achieved in either one application or in subsequent applications.

In a related embodiment of the invention, the energy transfer device also comprises a portion that is constructed with a material that is not able to be heated and/or that does not increase its temperature substantially, in order to protect the skin in the insertion point. In another embodiment of the invention, the heating element covers a majority of the body of the device except for the base, which is made of a non-heating element that does not increase its temperature substantially. This is of particular importance since the base may stay in contact with the epidermis before, during and after the application procedure and create undesired burns in the surrounding areas when heat is being applied to the dermis layer of the skin.

Further, in other embodiments of the invention, and in order to achieve more intense treatments with shorter times and higher temperatures, the energy transfer device may comprise laser, radiofrequency, or ultrasound application elements.

The apparatus of the invention provides a linear tensing effect through controlled heating of the dermis layer of the skin and may not require the technician to manipulate the device and move it through the skin. Movement through the skin can cause uneven results in the zone where the heat is applied such as in the aforementioned moving irradiation probe techniques.

The apparatus of the present invention generates a lineal tensing effect on the skin by applying heat to the dermis layer of the skin through a low cost, short duration, and painless method, wherein the apparatus also applies anesthetics through the same device, as well as allowing the application of other treatment fluids such as anti-inflammatory, anti-bruising, and/or collagen inductors that are applied in the same location of the treatment, representing an incomparable advantage and new feature compared to current treatments. Further, the apparatus can be used several times in the same spot to repeat the treatment as needed, which also cannot be achieved by current commercial apparatus.

Accordingly, the present invention provides a minimally invasive cosmetic procedure for improving the laxity of the skin and to delay the signs of aging in humans. In particular, the invention provides an innovative low cost, short time, and painless minimally invasive device and method for tightening sagging skin by means of linear tensing and stimulation of collagen production, wherein the apparatus not only allows to apply direct heat in the dermis layer of the skin, but also allows to administer an anesthetic and optionally additional treatment fluids such as anti-inflammatory, anti-bruising, or collagen induction fluids through the same apparatus.

In a very innovative manner, and in a completely different configuration than all known current and previous treatments performed commercially around the world, the energy transfer device in accordance with the present invention comprises a hollow device, wherein the device includes at least one orifice through which several substances can be inserted directly into the dermis layer of the patient. This feature has several advantages over current treatment apparatus and methods, and creates a completely new field of skin tightening processes that can achieve lower treatment times, less painful experiences, and a more controlled tensing effect of the skin. In an embodiment of the invention, the energy transfer device comprises a plurality of orifices to administer the anesthetic or other treatment fluids.

The energy transfer device further includes a temperature sensor located in the device that measures the temperature of the dermis immediately in contact with the energy transfer device. Therefore, this sensor allows the technician to ascertain the moment that the desired temperature is reached and to control the amount of time that said temperature will be maintained in order to provide the desired shrinkage rates. The sensor can be selected from thermocouples, thermistors, infrared sensors and thermometers, among others.

An external generation device is also provided as part of the apparatus of the present invention, wherein the external generation device is configured to provide the type of energy required to allow the energy transfer device to apply heat in the dermis layer.

In an embodiment of the invention where a heating element is used within the energy transfer device, the external generation device is configured to generate a magnetic field by generating high-frequency alternating electrical currents from 500 KHz to 10 MHz, in order to provide induction heating to the electromagnetically inducible material of the heating element.

In another embodiment of the invention where a heating element is used within the energy transfer device, the external generation device generates an electric current that is transferred to the heating element of the energy transfer device through a physical connection, such as a cable, wire, conductive cord, among others.

In other embodiments of the invention where the energy transfer device comprises a laser, ultrasound, or radiofrequency applicators, the external generation device is suitably configured to provide the required energy for such application, allowing to power the laser, ultrasound, or radiofrequency applicator located in the energy transfer device.

The apparatus of the invention can further comprise an external temperature control device configured to detect the temperature reached in the zone of the skin where the device is inserted. The external temperature control device comprises infrared cameras, among others.

In addition, the apparatus of the invention can further comprise cooling means that allow lowering the temperature of the device upon applying a high temperature to the patient's skin and that can operate while the procedure is being performed. In an embodiment, the cooling means is a device that applies cold air that allows for a rapid cooling of the skin upon application of temperature to the dermis. In other alternative embodiments, the cooling means is an external device or element that lowers the temperature of the skin such cold compresses or through the use of cold fluids. In another embodiment of the invention, a cooling fluid can be administered through the device or put in direct contact with the dermis or epidermis while inserted in the skin, to achieve localized cooling of such treatment area. In a further embodiment of the invention, the skin can be cooled off naturally over time.

### Description Of The Method

The present invention aims to provide a low cost minimally invasive linear tensing cosmetic method for delaying the signs of aging on a patient's skin, where the method comprises providing an energy transfer device for applying direct heat to the dermis. The energy transfer device is inserted into a dermal layer of a patient's skin, wherein the energy transfer device has an elongated shape like a hollow cannula or needle in order to allow introducing a fluid substance through the same. The energy transfer device is inserted into the patient's skin up to a length based on the area of the body where the treatment is being performed.

An external generation device is also provided as part of the apparatus according to the present invention, wherein the device is configured to provide the required type of energy according to the type of heating performed by the energy transfer device. In embodiments where the energy transfer device includes a heating element that increases its temperature to provide direct heating to the dermis through conduction, the external generation device is configured to generate an electric current or a magnetic field that allows increasing the temperature of the heating element located within the energy transfer device. In other embodiments of the invention, the external generation device allows powering a laser, ultrasound, or radiofrequency applicator located in the energy transfer device.

The method of the present invention requires the application of heat in the dermis layer of the skin through the energy transfer device, and comprises controlling the temperature and time of the heat application so that the dermis layer that is in direct contact with the energy transfer device achieves a temperature of between 45 and 75 degrees Celsius for a time range of up to 10 minutes.

The temperature of the dermis layer in immediate direct contact with the energy transfer device is measured through a temperature sensor located within the energy transfer device.

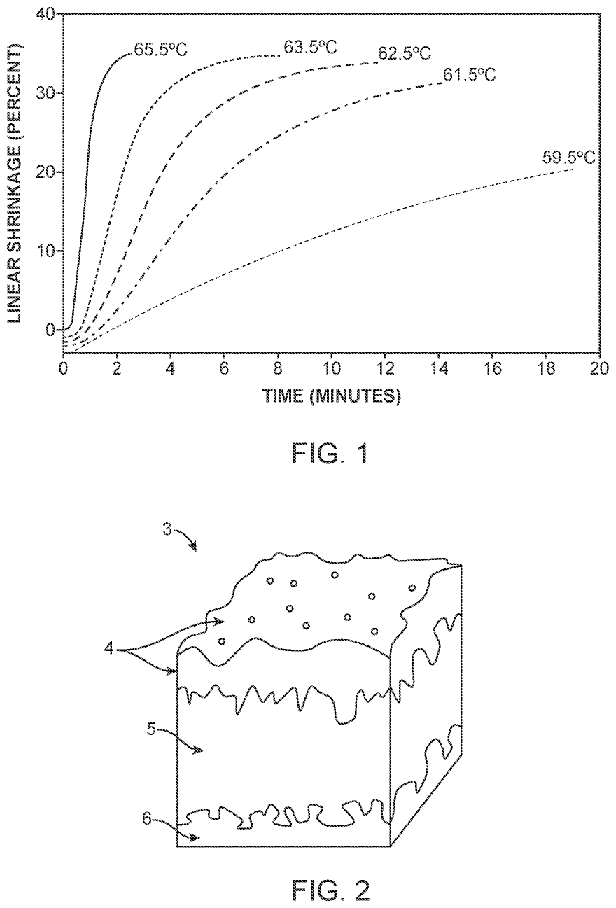

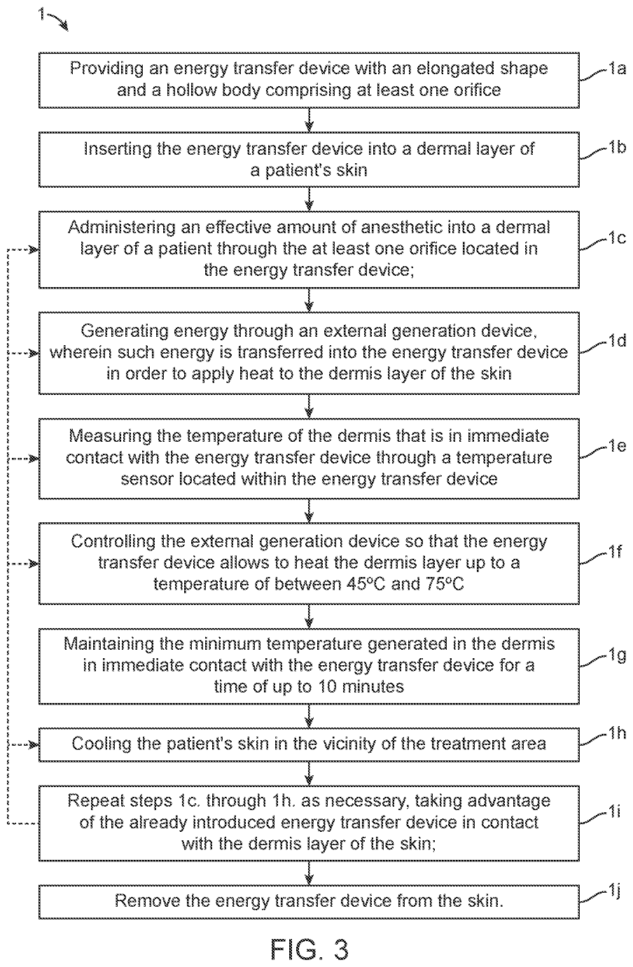

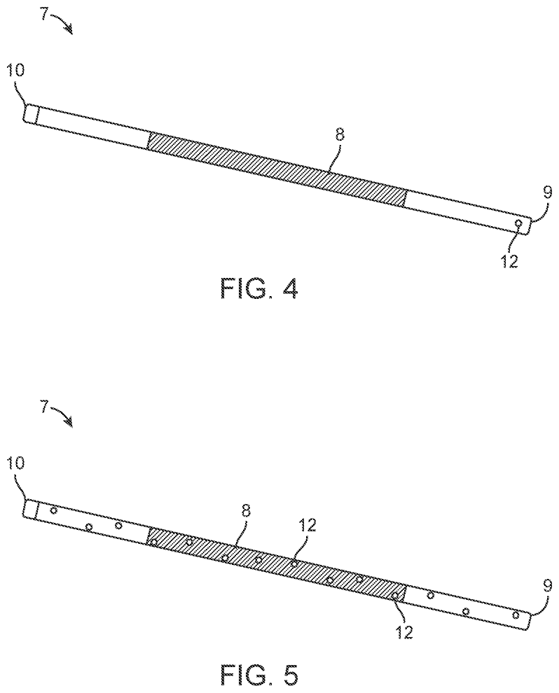

As seen in FIG. 1, previous experimental studies performed over the years have found that shrinking of collagen in the dermis depends on the applied temperature and the application time, where for example in one specific type of skin, a temperature of 62.5° C. maintained during about 10 minutes generates a 30% linear shrinkage, wherein maintaining 65.5° C. for about 1 minute generates a 20% linear shrinkage. In comparison, the same 20% can be achieved by maintaining 59.5° C. for about 20 minutes.

The method of the present invention requires the application of heat in the dermis layer of the skin through the energy transfer device, comprising controlling the temperature and time of the heating element located within the energy transfer device so that the dermis layer that is in direct contact with the energy transfer device achieves a shrinkage rate of between 5-35%.

Also as an important and innovative feature, the method of the present invention allows evaluating the shrinkage rate and aesthetic view of the lineal tensing of the skin in real time, allowing the technician to determine if the treatment needs to be repeated in the same location and to determine the temperature and treatment time to be applied on a specific patient. This is an important feature as current techniques such as microneedling do not allow visually evaluating the shrinkage rate but those treatments have to wait several weeks to provide the full effect. None of the current commercially applied methods allow to visualize and evaluate the lineal shrinkage of the skin in real time in order to determine next steps of the treatment.

The method of the present invention can be separated into two main areas, one where the treatment is intense and aims at producing high temperature applications during short periods of time (of less than 5 seconds), and the other where the treatment is less intense and aims at producing lower temperature applications for longer time periods (of more than 5 seconds and up to 10 minutes). Both treatment intensities depend on the energy transfer device and if it comprises a heating element (heated through electric conduction or electromagnetism) or other heat applicators such as lasers, ultrasound, or radiofrequency applicators.

In certain embodiments, and dependent on the specific conditions of the patient and the practitioner's technique, a shrinkage rate of between 5-35% can be achieved by applying a temperature of between 45 and 75 degrees Celsius for a time range depending on the treatment intensity, with a maximum application time of 10 minutes, which can be achieved in either one application or in subsequent applications.

As a very innovative and completely new feature of the present invention, the energy transfer device comprises at least one orifice through which an effective amount of an anesthetic or other substances is administered directly into the dermis layer during the treatment process, using the same apparatus for heating the dermis and generating a lineal tensing effect as well as for administering the anesthetic. Generally, the orifice is located within the body or tip of the energy transfer device. For example, an orifice located in the body or tip of the energy transfer device allows for a localized application of the anesthetic in the precise area where the application of temperature will take place. This is an advantageous feature of the invention compared to other known techniques which apply a sedative in random areas of the treatment location (e.g., a patient's face for example) causing unnecessary sedation and numbness in addition to the pain and discomfort during the anesthetic procedure.

In addition, and as an innovative and surprising effect, the at least one orifice also allows administering treatment fluids to the dermis layer of the patient after the procedure has been performed, wherein the treatment fluids are selected from the group comprising collagen and/or collagen inductors including but not limited to calcium hydroxyapatite, poly-L-lactic acid, and hyaluronic acid, an anti-inflammatory substance, an anti-bruising substance, and other type of substances that can help in the treatment and/or recovery of the treated zone.

The method according to the principles of the present invention requires at least one energy transfer device that is inserted into the skin and remains inserted during the treatment process. Advantageously, the method also allows for the insertion of many energy transfer devices into the dermis layer of the skin, and afterwards the heating and/or fluid administration processes can be performed simultaneously on at least two of the inserted energy transfer devices. The use of inserted devices allows for performing longer heat application periods at relatively low temperatures. In the case that multiple energy transfer devices are inserted, the apparatus is designed and configured to allow their simultaneous operation.

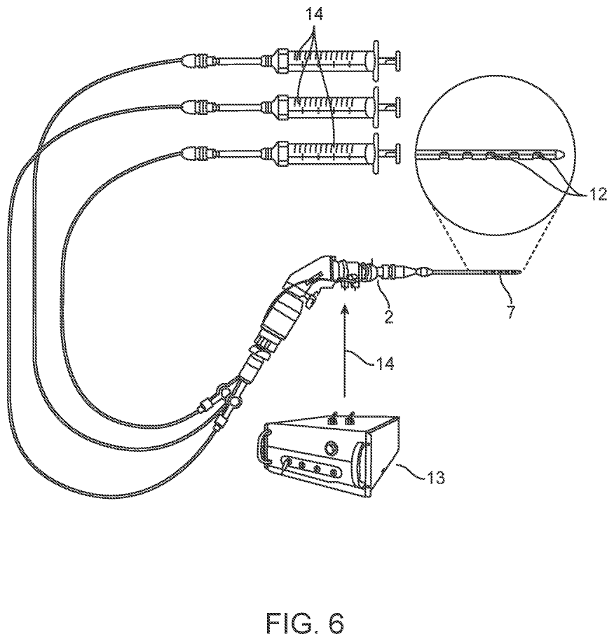

In another embodiment of the invention, the energy transfer device is located in a handheld device that is used in a specific treatment zone within the skin once at a time. As seen in FIG. 06, a handheld device (2) is shown, wherein the handheld device comprises an energy transfer device (7) with a plurality of orifices (12). The external generation device (13) is also shown, as well as fluid storage elements (14) that are connected to the energy transfer device in order to allow their administration to the dermis layer of the skin. The external generation device transfers the energy (shown as arrow 14 in the Figure) to the energy transfer device either by a physical connection, such as cables or similar means, or through induction. This device allows performing shorter heat application periods at relatively higher temperatures.

In another embodiment of the invention, the energy transfer device comprises a plurality of orifices distributed through its body and/or tip in order to administer the anesthetics and optionally other treatment fluids during the treatment process.

In an embodiment of the invention, the use of anesthetics such as lidocaine (mixed or not with epinephrine), conductive fluids, or other substances that would improve patient comfort or that would result in an improved post procedure cosmetic appearance of the tissue is preferred. A solution having a lidocaine concentration between 0.1% to 2% (preferably 0.25%), preferably mixed with epinephrine diluted to 1:100,000 to 1:500,000 (preferably 1:400,000) is preferred. Diluted lidocaine without epinephrine can also be used to suppress pain and improve patient comfort.

The method of the present invention, in an innovative manner, provides for repetition at the treatment site as many times as may be needed. The method of the present invention can repeated in the same treatment spot as required, without having to re-introduce a device. This is important as when high temperatures are applied to the skin, serious burns may result, or even nerves can be damaged, and therefore by allowing to repeat the treatment in the same spot, lower temperatures can be used in separate times, minimizing such risks. For example, the energy transfer device is used to heat the dermis so that the skin reaches an average temperature of within 45 and 75° C. as a maximum, then the skin area where the energy transfer device is inserted is cooled off, and then the heating element is again heated and increases its temperature and therefore allows heating the same dermis area to achieve a temperature of within 45 and 75° C. for a predetermined period of time. Suitable cooling means comprise cold air, ice, cold compresses, or natural cooling.

In a preferred embodiment of the invention, and before removing the energy transfer device from the patients skin, the energy transfer device can be cooled down through cooling means or naturally, and additional anti-inflammatory, anti-bruising, or skin tightening treatment substances, such as collagen inductors, are applied to the dermis through the orifices located within the device, comprising collagen and/or collagen inductors including but not limited to calcium hydroxyapatite, poly-L-lactic acid, and hyaluronic acid, an anti-inflammatory and other type of substances that can help in the treatment and/or recovery of the treated zone.

In another embodiment of the invention, the anti-inflammatory, anti-bruising, and/or skin tightening treatment substances such as collagen inductors are applied simultaneously with the anesthetic during the treatment through the apparatus of the present invention.

The method is finalized when the energy transfer device is removed from the skin.

The treatment of the present invention can be applied in different parts of the body, comprising not only the face and neck but also the rest of the body.

The device of the present invention therefore solves majority of the technical drawbacks of the techniques known from the state of the art as summarized in the following table.

TABLE 1
Comparison from Present Invention
with Microneedling Techniques

| Feature | Microneedling | Present Invention |
|---|---|---|
| Topical Anesthetic of | Usually applied | Not necessary |
| 30-45 minutes | | |
| Multiple injection with | About 60 points | Only one needed |
| anesthetic (lidocaine) | of application | per each energy |
| | for conventional | transfer device |
| | treatment | insertion |
| Certainty of | Not homogeneous, | Yes - applied |
| anesthesia | creates painful | precisely in the |
| | insertion areas | line of treatment |
| Punctures for | More than 200 total | Not needed |
| irradiating heat | punctures | |
| Time | Approximately 2 | Less than |
| | hours including | 40 minutes |
| | topic anesthesia | |
| Cost of equipment and | High | Low |
| treatment | | |
| Lineal tensing | No | Yes |
| Possibility of repeating | Not described | Possible |
| treatment in exactly same | | |
| point | | |
| Application of anesthetic | No | Yes |
| with same heating | | |
| apparatus | | |
| Application of anti- | No | Yes |
| inflammatory and | | |
| optionally collagen | | |
| inductors in precise | | |
| treatment area with | | |
| the same heating | | |
| apparatus | | |
| Inflammation and bruising | Mainly in all face | Limited to the lines |
| | and neck (when | of application and |
| | performed in that | allows to apply anti- |
| | location) | inflammatory in the |
| | | same treatment spot |
| Allows applying different | No | Yes |
| combinations of | | |
| temperature and time | | |
| in the same treatment | | |
| location | | |
| Allows to visualize lineal | No | Yes |
| shrinkage rate in real time | | |

TABLE 2
Comparison from Present Invention with
Moving Irradiating Probe Techniques

| Feature | Microneedling | Present Invention |
|---|---|---|
| Topical Anesthetic of | Usually applied | Not necessary |
| 30-45 minutes | | |
| Multiple injection with | About 60 points | Only one needed per |
| anesthetic (lidocaine) | of application | each energy transfer |
| | for conventional | device insertion |
| | treatment | |
| Certainty of | Not homogeneous, | Yes - applied |
| anesthesia | creates generates | precisely in the |
| | painful insertion | line of treatment |
| | areas | |
| Time | Approximately 2 | Less than |
| | hours including | 40 minutes |
| | topic anesthesia | |
| Cost of equipment and | High | Low |
| treatment | | |
| Possibility of repeating | Not described | Possible |
| treatment in exactly same | | |
| point | | |
| Application of anesthetic | No | Yes |
| with same heating | | |
| apparatus | | |
| Application of anti- | No | Yes |
| inflammatory and | | |
| optionally collagen | | |
| inductors in precise | | |
| treatment area with | | |
| the same heating | | |
| apparatus | | |
| Inflammation and bruising | Mainly in all face | Limited to the lines |
| | and neck (when | of application, and |
| | performed in that | allows to apply anti- |
| | location) | inflammatory in the |
| | | same treatment spot |
| Possibility of damage to | Possible | Seldom possible |
| tissue and nerves through | | |
| moving heated apparatus | | |
| Allows applying different | No | Yes |
| combinations of | | |
| temperature and time | | |
| in the same treatment | | |
| location | | |
| Allows to visualize lineal | No | Yes |
| shrinkage rate in real time | | |

It is expressly intended that, wherever possible, the invention includes combinations of aspects of the various embodiments described herein or even combinations of the embodiments themselves. Accordingly, other components that embody the principles of this invention can be configured within the spirit and intent of this invention. The arrangement described herein is provided as only one example of an embodiment that incorporates and practices the principles of this invention. Other modifications and alterations are well within the knowledge of those skilled in the art and are to be included within the broad scope of the appended claims.

What is claimed is:

1. A low cost minimally invasive apparatus for tightening sagging skin by linear tensing and stimulation of collagen production, which allows to administer fluids such as anesthetics and optionally other treatment fluids through the same apparatus, comprising:
an insertable energy transfer device having an elongated shape, wherein the device has a hollow body, a tip, and a base and includes:
at least one orifice located in the body or tip of the energy transfer device, through which an effective amount of an anesthetic and optionally other treatment fluids can be administered into a dermal layer of a patient; and
a temperature sensor located in the energy transfer device that measures the temperature of the dermis in immediate contact with the energy transfer device; and
an external generation device configured to generate energy that is transferred to the energy transfer device.

2. The apparatus according to claim 1, wherein the body of the energy transfer device is at least 1 cm long.

3. The apparatus according to claim 1, wherein the body of the energy transfer device is a hollow cannula.

4. The apparatus according to claim 1, wherein the body of the energy transfer device is a needle or syringe.

5. The apparatus according to claim 1, wherein the body of the energy transfer device has a double wall configuration.

6. The apparatus according to claim 1, wherein the body of the energy transfer device comprises a textured surface to achieve focalized energy transfer into the dermis layer of the skin.

7. The apparatus according to claim 1, wherein the body of the energy transfer device comprises spiral or helicoidally configured protrusions that generate a spring-like effect on the skin when such spirals transfer energy into the dermis layer of the skin.

8. The apparatus according to claim 1, wherein the body of the energy transfer device comprises elements or materials located in the inside or embedded into the energy transfer device, and that have an helicoidally configured pattern that cause heating through such elements or materials and therefore generates a spring-like effect on the skin when such materials or elements transfer energy into the dermis layer of the skin.

9. The apparatus according to claim 1, wherein the body or tip of the energy transfer device comprises a plurality of orifices to administer a treatment fluid to the dermis layer of the skin.

10. The apparatus according to claim 1, wherein the treatment fluids comprise anesthetics, anti-inflammatory, anti-bruising, and/or collagen induction substances.

11. The apparatus according to claim 1, wherein the energy transfer device comprises a heating element covering at least one portion of the body and/or tip of the energy transfer device, so that such material is able to increase its temperature in a controlled manner.

12. The apparatus according to claim 11, wherein the portion of the body not covered with the heating element, is covered with a non-heating element that does not increase its temperature substantially to protect the insertion point of the skin.

13. The apparatus according to claim 11, wherein the heating element is constructed so that it increases its temperature in response to an electrical current passing through a resistance that is generated by the external generation device.

14. The apparatus according to claim 11, wherein the heating element is constructed so that it increases its temperature driven by electricity generated by the external generation device.

15. The apparatus according to claim 11, wherein the heating element is constructed so that it increases its temperature in response to an electromagnetic field generated by the external generation device.

16. The apparatus according to claim 1, wherein the energy transfer device comprises lasers, radiofrequency irradiation, or ultrasound applicators in order to heat the dermis layer of the skin.

17. The apparatus according to claim 1, wherein the energy transfer device is configured so that a portion of it acts as a thermal insulator in order to avoid the heat to be transmitted and conducted to other portions of the skin.

18. The apparatus according to claim 11, wherein the heating element covers the majority of the body of the device except for the base, which is made of a non-heating element that does not increase its temperature substantially.

19. The apparatus according to claim 1, wherein the energy transfer device is located in a handheld device that is used in a specific treatment zone within the skin once at a time and allows performing shorter heat application periods at relatively higher temperatures.

20. The apparatus according to claim 1, wherein the apparatus is designed and configured to allow the simultaneous operation of at least two energy transfer devices that are inserted into the dermis layer of the skin.

21. The apparatus according to claim 1, wherein an external temperature control device is provided, such that the external temperature control device is configured to measure the temperature reached in the zone of the skin area where the device is inserted.

22. The apparatus according to claim 1, wherein cooling means are provided in order to lower the temperature of the skin of the patient.

23. The apparatus according to claim 1, wherein the cooling means is selected from the group comprising cold air, ice, or cold compresses.

24. The apparatus according to claim 1, wherein the cooling means is a cooling fluid administered through the device or put in direct contact with the dermis or epidermis while inserted in the skin, to achieve localized cooling of such treatment area.

25. The apparatus according to claim 1, wherein the external generation device allows to generate energy that is transferred to the energy transfer device, selected from the list comprising an electromagnetic field generator, an electric current generator, a radiofrequency generator, a laser generator, or an ultrasound generator.

26. The apparatus according to claim 1, wherein the external generation device transfers the energy to the energy transfer device either by a physical connection, such as cables or similar means, or through induction.

27. A low cost minimally invasive apparatus for tightening sagging skin by linear tensing and stimulation of collagen production, which allows to administer fluids such as anesthetics and optionally other treatment fluids through the same apparatus, comprising:
an insertable energy transfer device having an elongated shape, wherein the device has a hollow body, a tip, and a base and includes:
a heating element covering at least one portion of the body and/or tip of the energy transfer device, so that such material is able to increase its temperature in a controlled manner;
at least one orifice located in the body or tip of the energy transfer device, through which an effective amount of an anesthetic and optionally other treatment fluids can be administered into a dermal layer of a patient; and
a temperature sensor located in the energy transfer device that measures the temperature of the dermis in immediate contact with the energy transfer device; and
an external generation device configured to generate energy that is transferred to the energy transfer device in order to increase the temperature of the heating element.

28. The apparatus according to claim 27, wherein the body of the energy transfer device is at least 1 cm long.

29. The apparatus according to claim 27, wherein the body of the energy transfer device is a hollow cannula.

30. The apparatus according to claim 27, wherein the body of the energy transfer device is a needle or syringe.

31. The apparatus according to claim 27, wherein the body of the energy transfer device has a double wall configuration.

32. The apparatus according to claim 27, wherein the body of the energy transfer device comprises a textured surface to achieve focalized energy transfer into the dermis layer of the skin.

33. The apparatus according to claim 27, wherein the body of the energy transfer device comprises spiral or helicoidally configured protrusions that generate a spring-like effect on the skin when such spirals transfer energy into the dermis layer of the skin.

34. The apparatus according to claim 27, wherein the body of the energy transfer device comprises elements or materials located in the inside or embedded into the energy transfer device, and that have an helicoidally configured pattern that cause heating through such elements or materials and therefore generates a spring-like effect on the skin when such materials or elements transfer energy into the dermis layer of the skin.

35. The apparatus according to claim 27, wherein the body or tip of the energy transfer device comprises a plurality of orifices to administer a treatment fluid to the dermis layer of the skin.

36. The apparatus according to claim 27, wherein the treatment fluids comprise anesthetics, anti-inflammatory, anti-bruising, and/or collagen induction substances.

37. The apparatus according to claim 27, wherein the portion of the body not covered with the heating element, is covered with a non-heating element that does not increase its temperature substantially to protect the insertion point of the skin.

38. The apparatus according to claim 27, wherein the heating element is constructed so that it increases its temperature in response to an electrical current passing through a resistance that is generated by the external generation device.

39. The apparatus according to claim 27, wherein the heating element is constructed so that it increases its temperature driven by electricity generated by the external generation device.

40. The apparatus according to claim 27, wherein the heating element is constructed so that it increases its temperature in response to an electromagnetic field generated by the external generation device.

41. The apparatus according to claim 27, wherein the energy transfer device is configured so that a portion of it acts as a thermal insulator in order to avoid the heat to be transmitted and conducted to other portions of the skin.

42. The apparatus according to claim 27, wherein the heating element covers the majority of the body of the device except for the base, which is made of a non-heating element that does not increase its temperature substantially.

43. The apparatus according to claim 27, wherein the energy transfer device is located in a handheld device that is used in a specific treatment zone within the skin once at a time and allows performing shorter heat application periods at relatively higher temperatures.

44. The apparatus according to claim 27, wherein the apparatus is designed and configured to allow the simultaneous operation of at least two energy transfer devices that are inserted into the dermis layer of the skin.

45. The apparatus according to claim 27, wherein an external temperature control device is provided, such that the external temperature control device is configured to measure the temperature reached in the zone of the skin area where the device is inserted.

46. The apparatus according to claim 27, wherein cooling means are provided in order to lower the temperature of the patient's skin.

47. The apparatus according to claim 27, wherein the cooling means is selected from the group comprising cold air, ice, or cold compresses.

48. The apparatus according to claim 27, wherein the cooling means is a cooling fluid administered through the device or put in direct contact with the dermis or epidermis while inserted in the skin, to achieve localized cooling of such treatment area.

49. The apparatus according to claim 27, wherein the external generation device allows to generate energy that is transferred to the energy transfer device, selected from the list comprising an electromagnetic field generator, an electric current generator, a radiofrequency generator, a laser generator, or an ultrasound generator.

50. The apparatus according to claim 27, wherein the external generation device transfers the energy to the energy transfer device either by a physical connection, such as cables or similar means, or through induction.

51. A low cost minimally invasive linear tensing cosmetic method for tightening sagging skin by linear tensing and stimulation of collagen production by applying heat to the dermis layer of the skin directly, which comprises the next steps:
a) providing an energy transfer device with an elongated shape and a hollow body, comprising at least one orifice located within the body or tip of the energy transfer device;
b) inserting the energy transfer device into a dermal layer of a patient's skin;
c) administering an effective amount of anesthetic into a dermal layer of a patient through the at least one orifice located in the body or tip of the energy transfer device;
d) generating energy through an external generation device, wherein such energy is transferred into the energy transfer device in order to apply heat to the dermis layer of the skin;
e) measuring the temperature of the dermis that is in immediate contact with the energy transfer device through a temperature sensor located within the energy transfer device;
f) controlling the external generation device so that the energy transfer device allows to heat the dermis layer up to a temperature of between 45° to 75° C.;
g) maintaining the minimum temperature generated in the dermis in immediate contact with the energy transfer device for a time of up to 10 minutes;
h) cooling the patient's skin in the vicinity of the treatment area;
i) repeating steps c. through h. as necessary, taking advantage of the already introduced energy transfer device in contact with the dermis layer of the skin; and
j) removing the energy transfer device from the skin.

52. The minimally invasive cosmetic method from claim 51, wherein at least a portion of the energy transfer device comprises a heating element that increases its temperature in a controlled manner.

53. The minimally invasive cosmetic method from claim 52, wherein the heating element of the energy transfer device is constructed so that it increases its temperature in response to energy generated by the external generation device.

54. The minimally invasive cosmetic method from claim 53, wherein the external generation device allows to generate energy that is transferred to the energy transfer device, selected from the list comprising an electromagnetic field generator, an electric current generator, a radiofrequency generator, a laser generator, or an ultrasound generator.

55. The minimally invasive cosmetic method from claim 51, wherein by controlling the temperature and time of the heat application from the energy transfer device, the dermis layer that is in direct contact with the energy transfer device achieves a shrinkage rate of between 5-35%.

56. The minimally invasive cosmetic method from claim 55, wherein the shrinkage rate can be achieved in one or multiple applications.

57. The minimally invasive cosmetic method from claim 51, wherein the technician is able to visualize and evaluate the lineal shrinkage of the skin in real time in order to determine next steps of the treatment.

58. The minimally invasive cosmetic method from claim 51, wherein two or more energy transfer devices are inserted into the dermis layer of the skin, and afterwards the heating and/or fluid administration processes can be performed simultaneously on at least two of the inserted energy transfer devices.

59. The minimally invasive cosmetic method from claim 51, wherein the anesthetic comprises a solution having a lidocaine concentration between 0.1% to 2%.

60. The minimally invasive cosmetic method from claim 51, wherein the anesthetic comprises a solution having a lidocaine concentration between 0.1% to 2% that is mixed with epinephrine diluted to 1:100,000 to 1:500,000.

61. The minimally invasive cosmetic method from claim 51, wherein the method comprises administering additional treatment substances through the at least one orifice in the energy transfer device.

62. The minimally invasive cosmetic method from claim 61, wherein the treatment substances comprise collagen and/or collagen inductors including but not limited to calcium hydroxyapatite, poly-L-lactic acid, and hyaluronic acid, an anti-inflammatory, an anti-bruising substance, and other type of substances that can help in the treatment and/or recovery of the treated zone.

63. The minimally invasive cosmetic method from claim 51, wherein the anesthetic is mixed with the treatment fluids and administered simultaneously through the energy transfer device.

64. The minimally invasive cosmetic method from claim 51, wherein the cooling of the skin is performed with cooling means that comprise cold air, ice, or cold compresses.

65. The minimally invasive cosmetic method from claim 51, wherein the cooling of the skin does not require external cooling means but is done naturally.

66. The minimally invasive cosmetic method from claim 51, wherein the cooling means is a cooling fluid administered through the device or put in direct contact with the dermis or epidermis while inserted in the skin, to achieve localized cooling of such treatment area.

67. The minimally invasive cosmetic method from claim 51, wherein the method can be applied to the face and neck, as well as to other body parts.

68. A low cost minimally invasive linear tensing cosmetic method for tightening sagging skin by linear tensing and stimulation of collagen production by applying heat to the dermis layer of the skin directly, which comprises the next steps:
a) providing an energy transfer device with an elongated shape and a hollow body, comprising at least one orifice located within the body or tip of the energy transfer device, wherein at least a portion of the energy transfer device comprises a heating element that increases its temperature in a controlled manner;
b) inserting the energy transfer device into a dermal layer of a patient's skin;
c) administering an effective amount of anesthetic into a dermal layer of a patient through the at least one orifice located in the body or tip of the energy transfer device;
d) generating energy through an external generation device, wherein such energy is transferred into the energy transfer device in order to apply heat to the dermis layer of the skin by increasing the temperature of the heating element;
e) measuring the temperature of the dermis that is in immediate contact with the energy transfer device through a temperature sensor located within the energy transfer device;
f) controlling the external generation device so that the energy transfer device allows to heat the dermis layer up to a temperature of between 45° to 75° C.;
g) maintaining the minimum temperature generated in the dermis in immediate contact with the energy transfer device for a time of up to 10 minutes;
h) cooling the patient's skin in the vicinity of the treatment area;
i) repeating steps c. through h. as necessary, taking advantage of the already introduced energy transfer device in contact with the dermis layer of the skin; and
j) removing the energy transfer device from the skin.

69. The minimally invasive cosmetic method from claim 68, wherein the heating element of the energy transfer device is constructed so that it increases its temperature in response to energy generated by the external generation device.

70. The minimally invasive cosmetic method from claim 68, wherein the external generation device allows to generate energy that is transferred to the energy transfer device, selected from the list comprising an electromagnetic field generator and an electric current generator.

71. The minimally invasive cosmetic method from claim 68, wherein by controlling the temperature and time of the heat application from the energy transfer device, the dermis layer that is in direct contact with the energy transfer device achieves a shrinkage rate of between 5-35%.

72. The minimally invasive cosmetic method from claim 71, wherein the shrinkage rate can be achieved in one or multiple applications.

73. The minimally invasive cosmetic method from claim 68, wherein the technician is able to visualize and evaluate the lineal shrinkage of the skin in real time in order to determine next steps of the treatment.

74. The minimally invasive cosmetic method from claim 68, wherein two or more energy transfer devices are inserted into the dermis layer of the skin, and afterwards the heating and/or fluid administration processes can be performed simultaneously on at least two of the inserted energy transfer devices.

75. The minimally invasive cosmetic method from claim 68, wherein the anesthetic comprises a solution having a lidocaine concentration between 0.1% to 2%.

76. The minimally invasive cosmetic method from claim 68, wherein the anesthetic comprises a solution having a lidocaine concentration between 0.1% to 2% that is mixed with epinephrine diluted to 1:100,000 to 1:500,000.

77. The minimally invasive cosmetic method from claim 68, wherein the method comprises administering additional treatment substances through the at least one orifice in the energy transfer device.

78. The minimally invasive cosmetic method from claim 77, wherein the treatment substances comprise collagen and/or collagen inductors including but not limited to calcium hydroxyapatite, poly-L-lactic acid, and hyaluronic acid, an anti-inflammatory, an anti-bruising substance, and other type of substances that can help in the treatment and/or recovery of the treated zone.

79. The minimally invasive cosmetic method from claim 68, wherein the anesthetic is mixed with the treatment fluids and administered simultaneously through the energy transfer device.

80. The minimally invasive cosmetic method from claim 68, wherein the cooling of the skin is performed with cooling means that comprise cold air, ice, or cold compresses.

81. The minimally invasive cosmetic method from claim 68, wherein the cooling of the skin does not require external cooling means but is done naturally.

82. The minimally invasive cosmetic method from claim 68, wherein the cooling means is a cooling fluid administered through the device or put in direct contact with the dermis or epidermis while inserted in the skin, to achieve localized cooling of such treatment area.

83. The minimally invasive cosmetic method from claim 68, wherein the method can be applied to the face and neck, as well as to other body parts.
